(19) United States
(12) Patent Application Publication
(10) Pub. No.: US 2013/0053697 A1
(43) Pub. Date: Feb. 28, 2013

(54) ULTRASOUND IMAGING SYSTEM, ULTRASOUND PROBE, AND METHOD OF REDUCING POWER CONSUMPTION

(71) Applicants: Stefan Holl, Zipf (AT); Thomas Holl, Zipf (AT); Thomas Rittenschober, Zipf (AT); Bernd R. Arminger, Zipf (AT)

(72) Inventors: Stefan Holl, Zipf (AT); Thomas Holl, Zipf (AT); Thomas Rittenschober, Zipf (AT); Bernd R. Arminger, Zipf (AT)

(73) Assignee: GENERAL ELECTRIC COMPANY, Schenectady, NY (US)

(21) Appl. No.: 13/214,922

(22) Filed: Aug. 22, 2011

(51) Int. Cl. A61B 8/00

(52) U.S. Cl. 600/459

(57) ABSTRACT

An ultrasound imaging system, an ultrasound probe, and a method for detecting motion of an ultrasound probe with a motion sensor attached to the probe. The ultrasound system, ultrasound probe, and method also include reducing a power consumption of the ultrasound probe in response to detecting no motion for a period of time with the motion sensor.

### FIELD OF THE INVENTION

This disclosure relates generally to an ultrasound probe, an ultrasound imaging system, and a method for reducing the power consumption of an ultrasound probe.

### BACKGROUND OF THE INVENTION

In the field of medical ultrasound imaging, a transducer array is typically used to transmit ultrasound energy into a patient and to detect reflected ultrasound energy from the patient. The ultrasound probe is typically coated with an ultrasound gel to ensure good acoustical coupling and placed on a patient in order to efficiently transmit and receive ultrasonic energy. Based on the energy and timing of the reflected ultrasound waves, it is possible to determine detailed information about the region inside the patient. The information may be used to generate images and/or quantitative data such as blood flow direction or rate of flow.

The transducer array within the ultrasound probe typically includes a number of transducer elements that change shape in response to the application of a voltage across the element. The transducer elements are typically some type of piezoelectric material, such as PZT. By rapidly switching the voltage across the transducer elements, and timing the firing of the elements, an ultrasound beam of a specific frequency may be generated. When actively scanning, i.e. transmitting and receiving ultrasonic energy, the ultrasound probe typically generates heat from various sources. First, the mechanical motion of the piezoelectric elements generates heat and, second, the electronics within the ultrasound probe tend to generate heat as well. All this heat from within the ultrasound probe may be conductively transferred to the patient, since the ultrasound probe is only separated from the patient by a thin layer of ultrasound gel.

It is undesirable to have overly high ultrasound probe temperatures due to concerns about comfort and safety for patients and clinicians. In most countries, regulatory agencies have established strict guidelines regarding allowable temperatures for ultrasound probes. Contact with an ultrasound probe that is too hot could be uncomfortable or dangerous for patients and/or clinicians. Newer ultrasound probe designs, particularly 2D array probes have additional elements and channels, and, therefore, generate even more heat than older ultrasound probe designs.

For these and other reasons, there is a need for an improved ultrasound probe and method for controlling the temperature of an ultrasound probe.

### BRIEF DESCRIPTION OF THE INVENTION

The above-mentioned shortcomings, disadvantages and problems are addressed herein which will be understood by reading and understanding the following specification.

In an embodiment, an ultrasound imaging system includes an ultrasound probe including a transducer array and a motion sensor. The motion sensor is adapted to detect motion of the ultrasound probe. The ultrasound imaging system also includes a processor connected to the motion sensor. The processor is configured to reduce a power consumption of the ultrasound probe in response to detecting no motion for a period of time with the motion sensor.

In another embodiment, an ultrasound probe includes a probe housing, a transducer array disposed in the probe housing, a motion sensor disposed in the probe housing, and a processor disposed in the probe housing. The motion sensor is adapted to detect motion of the ultrasound probe and the processor is configured to reduce a power consumption of the ultrasound probe in response to detecting no motion for a period of time with the motion sensor.

In another embodiment, a method of controlling an ultrasound probe includes receiving data from a motion sensor attached to the ultrasound probe, the data indicating that the ultrasound probe has been motionless for a period of time and reducing a power consumption of the ultrasound probe in response to receiving the data.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic representation of an ultrasound probe in accordance with an embodiment;

FIG. 3 a schematic representation of an ultrasound probe in accordance with an embodiment; and

FIG. 4 is a flow chart of a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

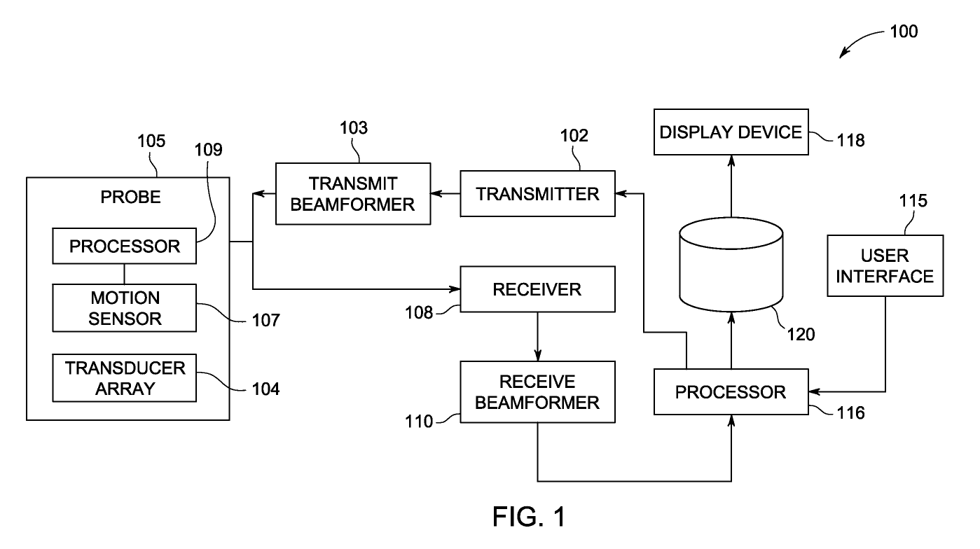

FIG. 1 is a schematic diagram of an ultrasound imaging system 100 in accordance with an embodiment. The ultrasound imaging system 100 includes a transmitter 102 that transmits a signal to a transmit beamformer 103 which in turn drives transducer elements (not shown) within a transducer array 104 to emit pulsed ultrasonic signals into a structure, such as a patient (not shown). An ultrasound probe 105 includes the transducer array 104, the transducer elements, a motion sensor 107, and a processor 109. The processor 109 may, for example, be a central processing unit, a microprocessor, a digital signal processor, or any other electrical component adapted for following logical instructions. A variety of geometries of transducer arrays may be used including 2D arrays, linear arrays, curved linear arrays, and convex arrays. Pulsed ultrasonic signals are back-scattered from structures in the body, like blood cells or muscular tissue, to produce echoes that return to the transducer array 104. The echoes are converted into electrical signals, or ultrasound data, by the transducer elements in the transducer array 104 and the electrical signals are received by a receiver 108. For purposes of this disclosure, the term ultrasound data may include data that was acquired and/or processed by an ultrasound imaging system. The electrical signals representing the received echoes are passed through a receive beamformer 110 that outputs ultrasound data. A user interface 115 may be used to control operation of the ultrasound imaging system 100, including, to control the input of patient data, to change a scanning or display parameter, and the like.

The ultrasound imaging system 100 also includes a processor 116 to process the ultrasound data and generate frames or images for display on a display device 118. The processor 116 may be adapted to perform one or more processing operations according to a plurality of selectable ultrasound modalities on the ultrasound data. The processor 116 may also be adapted to control the acquisition of ultrasound data with the ultrasound probe 105. The ultrasound data may be processed in real-time during a scanning session as the echo signals are received. For purposes of this disclosure, the term “real-time” is defined to include a process performed with no intentional lag or delay. An embodiment may update the displayed ultrasound image at a rate of more than 20 times per second. The images may be displayed as part of a live image. For purposes of this disclosure, the term “live image” is defined to include a dynamic image that updates as additional frames of ultrasound data are acquired. For example, ultrasound data may be acquired even as images are being generated based on previously acquired data while a live image is being displayed. Then, according to an embodiment, as additional ultrasound data are acquired, additional frames or images generated from more-recently acquired ultrasound data are sequentially displayed. Additionally or alternatively, the ultrasound data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the ultrasound signal while a second processor may be used to further process the data prior to displaying an image.

Still referring to FIG. 1, the ultrasound imaging system 100 may continuously acquire ultrasound data at a frame rate of, for example, 20 Hz to 150 Hz. However, other embodiments may acquire ultrasound data at a different rate. A memory 120 is included for storing processed frames of acquired ultrasound data that are not scheduled to be displayed immediately. In an exemplary embodiment, the memory 120 is of sufficient capacity to store at least several seconds worth of frames of ultrasound data. The frames of ultrasound data are stored in a manner to facilitate retrieval thereof according to its order or time of acquisition. As described hereinabove, the ultrasound data may be retrieved during the generation and display of a live image. The memory 120 may comprise any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring ultrasound data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well known by those skilled in the art and will therefore not be described in further detail.

In various embodiments of the present invention, ultrasound data may be processed by other or different mode-related modules (e.g., B-mode, Color Doppler, power Doppler, M-mode, spectral Doppler, anatomical M-mode, strain, strain rate, and the like) to form 2D or 3D data sets of image frames and the like. For example, one or more modules may generate B-mode, color Doppler, power Doppler, M-mode, anatomical M-mode, strain, strain rate, spectral Doppler image frames and combinations thereof, and the like. The image frames are stored and timing information indicating a time at which the image frame was acquired in memory may be recorded with each image frame. The modules may include, for example, a scan conversion module to perform scan conversion operations to convert the image frames from Polar to Cartesian coordinates. A video processor module may be provided that reads the image frames from a memory and displays the image frames in real time while a procedure is being carried out on a patient. A video processor module may store the image frames in an image memory, from which the images are read and displayed. The ultrasound imaging system 100 may be configured as a console system, a cart-based system, or a portable system, such as a hand-held or laptop-style system.

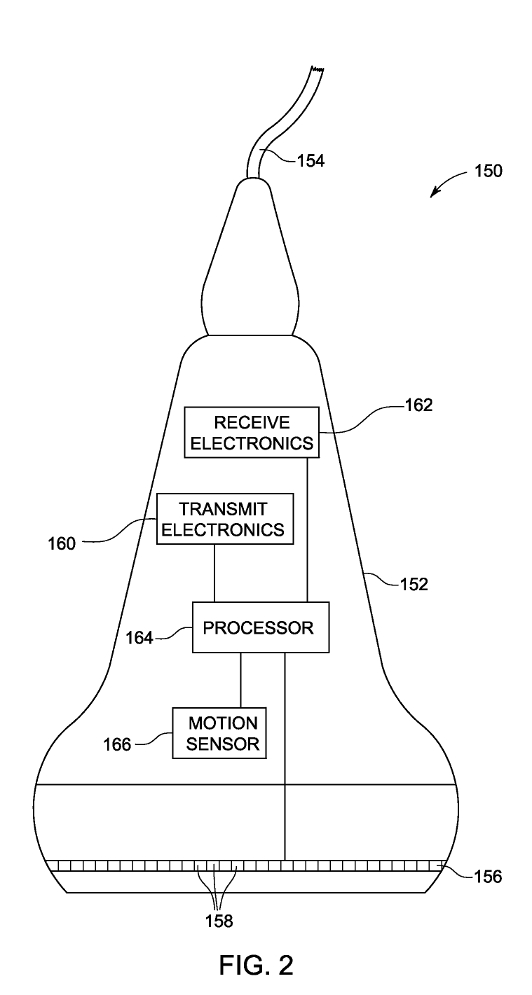

FIG. 2 is a schematic representation of an ultrasound probe 150 in accordance with an embodiment. The ultrasound probe 150 may be used in place of the ultrasound probe 105 in an ultrasound imaging system such as the ultrasound imaging system 100 shown in FIG. 1. The ultrasound probe 150 includes a probe housing 152. The probe housing 152 may be plastic according to an embodiment, and it may be shaped to allow for ergonomic use by a clinician. For example, the probe housing 152 may be shaped to be comfortably held between a clinician's thumb and index finger. A cord 154 is attached to the probe housing 152 and is used to transfer data between the ultrasound probe 150 and the rest of the ultrasound imaging system. According to an embodiment, the ultrasound probe 150 includes a transducer array 156 including a plurality of transducer elements 158. As described previously, the transducer elements 158 are configured to transmit and receive ultrasonic energy in order to form an image and/or acquire data of structures inside of a patient. The ultrasound probe 150 also includes transmit electronics 160, receive electronics 162, and a processor 164.

The transmit electronics 160 may include one or more electrical components that are used during the transmission of ultrasound energy. For example, the transmit electronics 160 may include one or more of a high-voltage-supply, a waveform generator, time-delay-line-elements, and post-amplifier-elements. In order to actively transmit an ultrasound beam into a patient, it may be necessary to bias the transmit electronics 160 at a non-zero voltage. The receive electronics 162 may include one or more of pre-amplifier elements, time-delay-line-elements, and a signal-summing-unit. In order to actively receive reflected ultrasound waves, it may be necessary to bias the receive electronics 162 at a non-zero voltage.

The ultrasound probe 150 also includes a motion sensor 166. According to an exemplary embodiment, the motion sensor 166 may include an accelerometer capable of detecting accelerations. For example, the motion sensor 166 may be a 3-axis accelerometer with 3 accelerometers mounted orthogonally to each other. This way, the 3-axis accelerometer is adapted to detect components of acceleration in any direction. It should be understood by those skilled in the art, that other embodiments may use other types of motion sensors. For example, embodiments may use a gyroscopic sensor as a motion sensor. A gimbaled gyroscope tends to remain spinning in the same orientation regardless of the position of the surrounding object, or ultrasound probe according to an embodiment. Therefore, by measuring displacement with respect to the spinning gyroscope, it is possible to determine if the ultrasound probe is moving or stationary. It should be appreciated that gyroscopes tend to precess while spinning. This precession may need to be taken into account when interpreting data from a gyroscopic sensor. According to other embodiments, other types of motions sensors may also be used. For example, electromagnetic sensors configured to detect the position of a sensor with respect to a static field may be used. Other types of motion sensors, including those using optical sensors, may also be used.

The motion sensor 107 is connected to the processor 109 in accordance with an embodiment. The processor 109 receives data from the motion sensor 107 and, based on these data, determines when the ultrasound probe 105 is in motion and when the ultrasound probe 105 is not in motion. According to another embodiment, the processor connected to the motion sensor may be positioned outside of the ultrasound probe. For example, a motion sensor may be connected to a processor, such as the processor 116, that is located outside of the ultrasound probe 105. According to an embodiment, the processor 116 may be within a console of an ultrasound imaging system. Therefore, according to other embodiments, the processor 116 or another processor located outside of the ultrasound probe 105 may receive data from the motion sensor and potentially control the deactivation of the ultrasound probe if the data indicates that the ultrasound probe 105 is not moving. The deactivation of the ultrasound probe will be described in more detail hereinafter.

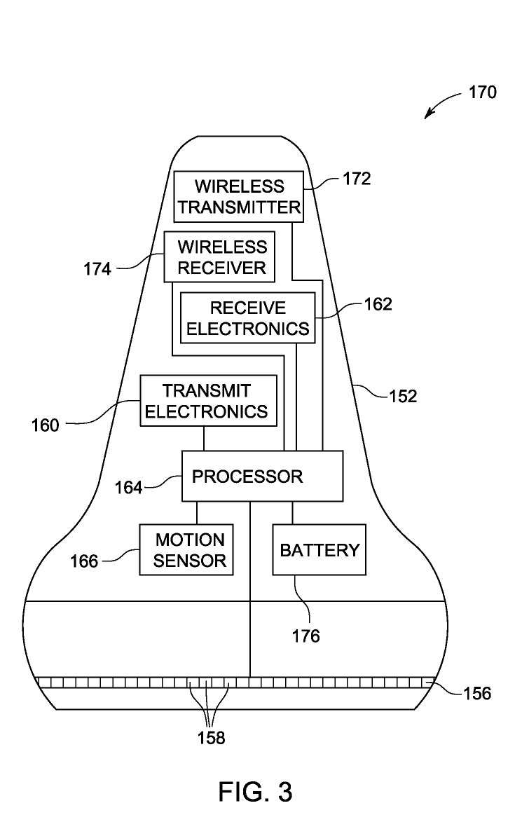

FIG. 3 is a schematic representation of an ultrasound probe 170 in accordance with an embodiment. The ultrasound probe 170 shares many components with the ultrasound probe 150 (shown in FIG. 2). For simplicity, common reference numbers will be used to identify components that are substantially the same between FIGS. 2 and 3. The ultrasound probe 170 also includes a wireless transmitter 172, a wireless receiver 174, and a battery 176. According to some embodiments, the wireless transmitter 172 and the wireless receiver 174 may be replaced by a single wireless transmitter/receiver (not shown). In FIG. 3, the wireless transmitter 172, the wireless receiver 174, and the battery 176 are all shown as connected to the processor 164. However, according to other embodiments, the components may be connected in a different manner. For example, the transmit electronics 160 may be directly connected to the transducer array 156 and the receive electronics 162 may be directly connected to the wireless transmitter 172.

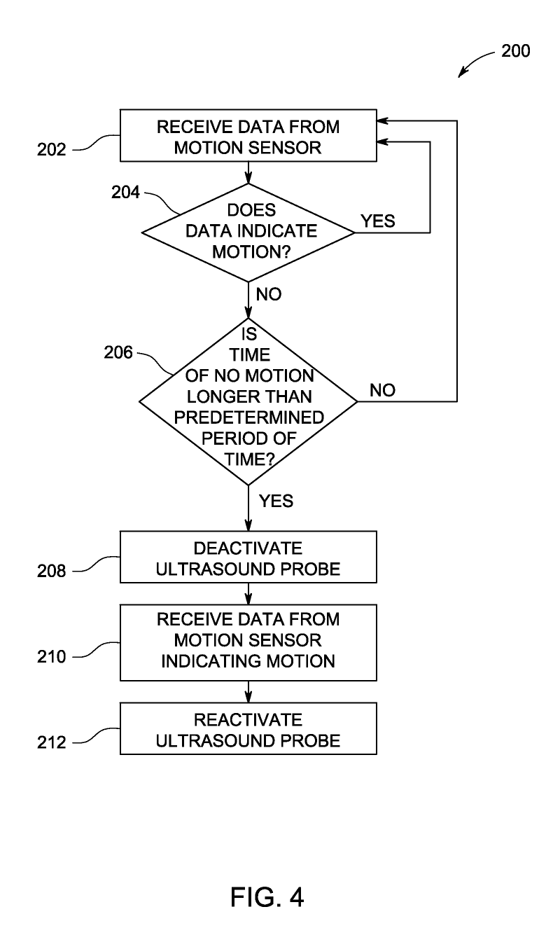

FIG. 4 is a flow chart of a method 200 in accordance with an embodiment. The individual blocks represent steps that may be performed in accordance with the method 200. The technical effect of the method 200 is the deactivation of the ultrasound probe in response to receiving data from a motion sensor indicating a period of no motion.

Referring to FIGS. 1 and 4, at step 202, the processor 109 receives data from the motion sensor 107. The motion sensor 107 may push data to the processor 109 at regular intervals or the motion sensor 107 may transmit data continuously in real-time. At step 204, the processor 109 determines if the data indicates that the ultrasound probe 105 is in motion. Typically, when in use, the ultrasound probe 105 would be in nearly constant motion. The motion sensor 107 is sensitive enough to detect even small movements that would occur even when a clinician is trying to hold the ultrasound probe 105 stationary. For example, if the data from the motion sensor 107 indicates that the ultrasound probe 105 is in motion, then the method returns to step 202 where additional data is received. However, if the data from the motion sensor 107 indicates that the ultrasound probe 105 is experiencing no motion, then the method 200 proceeds to step 206.

At step 206, the processor 109 determines if the period of time that the ultrasound probe 105 is motionless exceeds a predetermined period of time. For example, according to an embodiment, the predetermined period of time may be 2 seconds. If the processor 109 determines that the ultrasound probe 105 has been motionless for more than 2 seconds, then the method 200 advances to step 208. Other embodiments may use a predetermined period of time other than 2 seconds. Other embodiments may allow the period of time to be user configurable. In other words, the user or clinician may be able to select the most appropriate predetermined period of time. Additionally, some embodiments may have the actions of steps 204 and 206 combined into one step. For example, the processor 109 may allow the method to advance only if the period of time that ultrasound probe 105 has been motionless exceeds the predetermined period of time.

Next, at step 208, the processor 109 deactivates the ultrasound probe 105. According to an embodiment, the processor 109 may deactivate the ultrasound probe 105 by reducing the power consumption of the ultrasound probe 105. Reducing the power consumption of the ultrasound probe 105 is beneficial since it helps to decrease the external temperature of the ultrasound probe 105. This makes it easier to keep the ultrasound probe 105 within safe operating limits. Additionally, for embodiments where the ultrasound probe is wireless, such as the ultrasound probe 170 shown in FIG. 3, reducing the power consumption when the ultrasound probe is stationary and not in use helps to conserve battery 176 power. Additional information about various ways to deactivate the ultrasound probe 105 will be described in detail hereinafter.

According to an embodiment, the processor 109 continues to receive data from the motion sensor 107 during steps 206 and 208. However, once the ultrasound probe 105 has been moved, at step 210, the processor 109 will receive data from the motion sensor 107 indicating that the ultrasound probe 105 is back in motion. Then, at step 212, the processor reactivates the ultrasound probe 105 so that the clinician may again acquire ultrasound data.

As described previously, the processor 109 may deactivate the ultrasound probe at step 208. There are many different ways to deactivate the ultrasound probe at step 208. According to an embodiment, the process of deactivation may include a plurality of steps performed in a sequence. For example, a first stage of deactivation may include reducing the frame rate at which ultrasound data is acquired. Reducing the frame rate of the ultrasound data will conserve power, while allowing for the ultrasound probe 105 to be reactivated very quickly if movement is detected with the motion sensor 107. Next, after additional time without motion, the processor 109 may reduce or eliminate the voltage to the transmit electronics 160 (shown in FIG. 2). This will result in a further reduction in power usage. Next, after yet more time has passed without detecting motion with the motion sensor 107, the processor 109 may freeze the ultrasound probe 105. Freezing the ultrasound probe includes stopping the process of scanning, but keeping the ultrasound probe “alive” so that it is possible to resume scanning. For example, clock signals within the ultrasound probe may still be active, and voltages may still be applied to the transmit electronics 160 (shown in FIG. 2) and the receive electronics 162 (shown in FIG. 2). Freezing the ultrasound probe 105 reduces power consumption, since ultrasound energy is not actively transmitted, but since voltages are still applied to the transmit electronics 160 (shown in FIG. 2) and receive electronics 162 (shown in FIG. 2), there is very little lag time if it is desired to resume scanning with the ultrasound probe. Finally, if enough time has passed, it may be desirable to totally deselect the ultrasound probe. This may include turning off the voltages to all the electronic devices in the ultrasound probe. This final technique results in the maximum reduction of power consumption, but it also takes the most amount of time to reactivate the ultrasound probe. Therefore, it may only be desirable to totally deselect the ultrasound probe after a longer period of time, such as several minutes, has passed with the ultrasound probe experiencing no motion. According to other embodiments, only one or more of the stages of deactivation described above may be employed. According to other embodiments, one or more processors located outside of the probe may be used in place of processor 109 during the method 200. For example, a processor located on the ultrasound system outside of the probe, such as processor 116 may receive data from the motion sensor 107 and determine whether or not the probe should be deactivated during the method 200. Additionally, according to other embodiments, a processor outside of the ultrasound probe 105 may control the deactivation of the ultrasound probe 105.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An ultrasound imaging system comprising:
an ultrasound probe comprising a transducer array and a motion sensor, wherein the motion sensor is adapted to detect motion of the ultrasound probe; and
a processor connected to the motion sensor, wherein the processor is configured to reduce a power consumption of the ultrasound probe in response to detecting no motion for a period of time with the motion sensor.

2. The ultrasound imaging system of claim 1, wherein the motion sensor comprises an accelerometer.

3. The ultrasound imaging system of claim 1, wherein the motion sensor comprises a gyroscopic sensor.

4. The ultrasound imaging system of claim 1, wherein the processor is further configured to reduce the power consumption of the ultrasound probe by reducing a voltage to an electrical component in the ultrasound probe.

5. The ultrasound imaging system of claim 4, wherein the processor is further configured to reactivate the ultrasound probe by restoring the voltage to the electrical component in response to detecting motion from the motion sensor.

6. The ultrasound imaging system of claim 5, wherein the electrical component comprises a pre-amplifier element, a time-delay-line-element, or a signal-summing-unit.

7. An ultrasound probe comprising:
a probe housing;
a transducer array disposed in the probe housing;
a motion sensor disposed in the probe housing, wherein the motion sensor is adapted to detect motion of the ultrasound probe; and
a processor disposed in the probe housing, the processor being electrically coupled to the motion sensor, the processor configured to reduce a power consumption of the ultrasound probe in response to detecting no motion for a period of time with the motion sensor.

8. The ultrasound probe of claim 7, wherein the motion sensor comprises an accelerometer.

9. The ultrasound probe of claim 8, wherein the motion sensor comprises a 3-axis accelerometer.

10. The ultrasound probe of claim 7, wherein the motion sensor comprises a gyroscopic sensor.

11. The ultrasound probe of claim 7, wherein the processor is further configured to reduce the power consumption of the ultrasound probe by reducing a voltage to an electrical component in the ultrasound probe.

12. The ultrasound probe of claim 11, wherein the processor is further configured to reactivate the ultrasound probe by restoring the voltage to the electrical component in response to detecting motion from the motion sensor.

13. A method of controlling an ultrasound probe, the method comprising:
receiving data from a motion sensor attached to the ultrasound probe, the data indicating that the ultrasound probe has been motionless for a period of time; and
reducing a power consumption of the ultrasound probe in response to said receiving the data.

14. The method of claim 13, wherein said reducing the power consumption comprises reducing a transmit voltage.

15. The method of claim 13, wherein said reducing the power consumption comprises stopping a scanning function of the ultrasound probe.

16. The method of claim 15, further comprising detecting motion with the motion sensor after said stopping the scanning function of the ultrasound probe.

17. The method of claim 16, further comprising resuming the scanning function of the ultrasound probe in response to said detecting the motion with the motion sensor.

18. The method of claim 13, wherein said reducing the power consumption comprises switching off all the voltages in the ultrasound probe.

19. The method of claim 13, wherein said receiving data comprises receiving data from an accelerometer.

20. The method of claim 13, wherein the period of time comprises 2 or more seconds.
